(19) United States
(12) Patent Application Publication
(10) Pub. No.: US 2005/0123902 A1
(43) Pub. Date: Jun. 9, 2005

(54) Human papillomavirus inhibitors

(75) Inventors: Patricio I. Meneses, Philadelphia, PA (US); Angela N. Koehler, Cambridge, MA (US); Jason C. Wong, Oberlin, OH (US); Peter M. Howley, Wellesley, MA (US); Stuart L. Schreiber, Boston, MA (US)

Correspondence Address: CHOATE, HALL & STEWART LLP, EXCHANGE PLACE, 53 STATE STREET, BOSTON, MA, 02109, US

(73) Assignee: President and Fellows of Harvard College

(21) Appl. No.: 10/851,407

(22) Filed: May 21, 2004

Related U.S. Application Data
(60) Provisional application No. 60/472,261, filed on May 21, 2003.

(51) Int. Cl. C12Q 1/70; A61K 31/41; C07K 7/08

(52) U.S. Cl. 435/5; 530/326; 514/381

(57) ABSTRACT

The present invention provides systems for identifying anti-viral agents. In particular, the invention encompasses reagents and strategies for identifying agents that inhibit or disrupt key protein-protein interactions that are important in the life cycle of papillomaviruses. The invention allows identification, production, and/or use of agents that reduce or inhibit the replication of HPV by inhibiting (e.g., precluding, reversing, or disrupting) the formation of the E1-E2 protein-protein complex. The invention also provides specific inhibitory agents, pharmaceutical compositions, and methods of using these inhibitors and pharmaceutical compositions for inhibiting viral replication in vitro. Methods are also described for the treatment and prevention of HPV infections and HPV-related diseases in patients.

### RELATED APPLICATION

This application claims priority to Provisional Patent Application No. 60/472,261, filed on May 21, 2003, which is incorporated herein by reference in its entirety.

### GOVERNMENT INTERESTS

The work described herein was funded by the National Institutes of Health (Grant Nos. 5RO1CA77385 and R01 GM38627-17) and by the National Cancer Institute (ICCB MTL). The United States government may have certain rights in the invention.

### BACKGROUND OF THE INVENTION

Papillomaviruses (PVs) are small, circular double-stranded DNA viruses that cause benign epithelial and fibroepithelial lesions (commonly called warts) in a wide variety of species. More than 70 strains are known to infect humans (E. M. de Villiers, Curr. Top. Microb. Immunol. 1994, 186: 1-12; H. Zur Hausen and E. M. de Villiers, Annu. Rev. Microbiol. 1994, 48: 427-447; and P. M. Howley, “Papillomavirinae: The viruses and their replication” in B. N. Fields et al. (Eds.), Fields Virology, 3^{rd} Ed., 1996, Raven: Philadelphia, Pa. pp. 2045-2076). Human papillomaviruses (HPVs) are broadly grouped into cutaneous and mucosal types, based on the clinical location of the lesion. Although some overlap exists, most papillomaviruses have distinct anatomical predilection, infecting only a particular epidermal site either inside or outside the body.

Genito-mucosal lesions are the most clinically significant diseases associated with HPV. An estimated 24 million Americans are infected with genital HPV and between 0.5 and 1.0 million new cases are diagnosed annually (K. R. Beutner et al., Clin. Infect. Dis. 1998, 27: 796-806). Genital HPV infection is one of the most common sexually transmitted diseases (STDs), behind Chlamydia and Gonorrhea, and the most frequent viral STD (E. L. Franco et al., Can. Med. Assoc. J. 2001, 164: 1017-1025). Furthermore, certain types of Human Papillomavirus, categorized as high-risk HPVs, are implicated in the development of cervical dysplasia and cervical cancer (M. H. Schiffman et al., J. Natl. Cancer Inst. 1993, 85: 958-964). In developing countries, cervical cancer is the most frequent female malignancy, accounting for about a quarter of all cancers in women. Annually, approximately 500,000 new cases of invasive carcinomas of the cervix are diagnosed worldwide and the disease causes approximately 200,000 deaths (D. M. Parkin et al., Int. J. Cancer 1999, 80: 827-841; and P. Pisani et al., Int. J. Cancer, 1999, 83: 18-29). Epidemiological studies showed that virtually all cervical cancers contain the genes of high-risk HPVs (including HPV-16, -18, -31, and -33), with a prevalence of HPV-16, which is detected in 50 to 70% of cervical tumors (H. zur Hausen, Nature, 1991, 254: 1167-1173).

Despite the high incidence of genital HPV infection and its association with malignant diseases, there is no effective antiviral therapy for HPV infection (L. M. Cowsert, Intervirol. 1994, 37: 226-230). Current therapeutic approaches involve the removal of warts by surgery or necrotization using cryo-, electron, or laser cauterization. Although these techniques destroy the warty growths, they usually do not completely eradicate the virus, which leads to high recurrence rates. Medicinal methods based on the administration of podophyllotoxin or interferon are only weakly efficient and are also associated with strong side effects and/or after-effects. Identification and design of selective chemotherapeutic agents to control HPV is all the more difficult given that papillomaviruses do not encode their own DNA polymerase and rely upon host cellular machinery for replication.

More specifically, primary infection with papillomavirus occurs in the basal cell layer of the squamous epithelium. Contrary to most other viral pathogens, PVs are non-lytic viruses: they maintain their DNA genome at a low copy number until the infected cell migrates to the upper layer of the epithelium. There, as the infected cell differentiates into a keratinocyte, the viral DNA genome is amplified, structural viral proteins are expressed, and infectious virions are produced. All known PVs express similar early genes (E1-E8), several of which code for proteins with regulatory functions. The early E1 and E2 proteins are involved in viral DNA replication, while the other early proteins play important roles in processes such as regulation of the cell cycle. The E2 protein directs replication by binding to viral DNA and to the E1 protein with high affinity to form a viral replication complex. Cellular components needed for viral replication have been shown to bind and be recruited to the viral origin of replication either by E1 or E2 (C. M. Chiang et al., J. Virol. 1992, 66: 5224-5231; C. M. Chiang et al., Proc. Natl. Acad. Sci. USA 1992, 89: 5799-5803; M. Ustav and A. Stenlund, EMBO J. 1991, 10: 449-457).

Current strategies aimed at preventing the spread of a papillomavirus involve interfering with the binding of the viral capsid molecules to cellular receptor(s). In a different approach, work has been directed at disrupting the life cycle of papillomaviruses by disrupting viral DNA replication. The hypothesis is that if viral DNA replication is disrupted, infectious virions will not be produced, which will result in a reduction of the spread of the virus, and consequently in a reduction of the incidence of cutaneous warts and cervical carcinomas associated with high-risk HPV infection.

Several different strategies have been used so far to disrupt viral DNA replication. One approach is to block the expression of the E2 protein. This has been successfully achieved using antisense oligonucleotides targeted at E2 MRNA (L. Cowsert et al., Antimicrob. Agents Chemother. 1993, 37: 171-177; WO 93/20095). Another strategy is to directly target the E2 protein itself. In this regard, truncated forms of the E2 protein containing the DNA-binding domain have been shown to act as trans-activating repressors by blocking the homodimerization of E2 (R. B. Pepinsky et al., DNA Cell Biol. 1994, 13: 1011-1019; U.S. Pat. No. 5,219,990). Similarly, modified forms of the E2 protein that have a high affinity for E2 binding sites on the papillomavirus DNA have been shown to prevent the native E2 protein from binding to viral DNA and therefore to inhibit viral DNA replication (EP 0 302 758).

The development of therapeutic agents against HPV can also make use of the fact that formation of the E1-E2 complex is necessary for the stimulation of viral DNA replication. Some of the present inventors have demonstrated the feasibility of this approach by showing that a 15-mer peptide corresponding to a region of the HPV-16 E2 protein was capable of preventing the E1 and E2 proteins from binding, and most importantly could inhibit papillomavirus DNA replication in vitro (H. Sakai et al., J. Virol. 1996, 70: 1602-1611; and H. Kasukawa et al., J. Virol. 1998, 72: 8166-8173). Using this observation, the authors of this work have designed and developed E2-derived peptides and E2-based synthetic peptidomimetics exhibiting such inhibiting properties and have shown that these compounds could be used as anti-viral agents to control HPV infection (U.S. Pat. Nos. 6,399,075 and 6,432,926).

Although these studies demonstrate that the E2 protein can indeed serve as viable target for the development of therapeutics against papillomaviruses, they all use large biomolecules (molecular weight>1000) to achieve this goal. Rare are the studies directed at identifying small molecules as potential PV anti-viral agents (P. J. Hajduk et al., J. Med. Chem. 1997, 40: 3144-3150). Clearly, there remains a need to identify and develop simpler, preferably cell-permeable, small molecule therapeutics that can be used for the treatment and prevention of papillomavirus-induced clinical conditions.

### SUMMARY OF THE INVENTION

The present invention provides systems for identifying anti-viral agents. In particular, the invention encompasses reagents and strategies for identifying agents that inhibit or disrupt protein-protein interactions that are important in the viral life cycle. In certain preferred embodiments, the invention allows identification, production, and/or use of agents that inhibit a human papillomavirus, for example, by inhibiting (e.g., precluding, reversing, or disrupting) the formation of the E1-E2 protein-protein complex.

In one aspect, the invention provides a system including an interacting peptide that comprises a portion of a viral interacting protein, and a specificity peptide that is identical in amino acid sequence to the interacting peptide except that it contains a mutation or alteration that reduces or destroys its ability to bind to the interacting protein's partner. In certain embodiments, binding interactions between the viral interacting protein and the interacting protein's partner are important in the life cycle of HPV. Preferably, the viral interacting protein is the E1 protein or E2 protein.

In another aspect, the invention provides methods for identifying anti-viral agents by contacting candidate compounds or factors with both the interacting and specificity peptides; those compounds or factors that bind to the interacting peptide and not to the specificity peptide are classified as inhibitory agents. In certain embodiments, the inventive methods are used for identifying agents that inhibit or disrupt certain protein-protein interactions that are important in the life cycle of HPV. Preferably, the inventive methods are used for identifying agents that inhibit HPV by inhibiting (e.g., precluding, reversing, or disrupting) the formation of the E1-E2 protein-protein complex. In other embodiments, the inventive methods are used for testing small molecules. In still other embodiments, the inventive methods are used for screening small molecule libraries.

In another aspect, the present invention provides inhibitory agents identified by the screening methods described. In certain embodiments, inhibitory agents are small molecules or chemical derivatives of small molecules identified by the inventive methods. Preferably, inhibitory agents of the invention are small molecules that inhibit HPV. More preferably, inhibitory agents of the invention are small molecules that inhibit HPV by inhibiting (e.g., precluding, reversing, or disrupting) the formation of the E1-E2 protein-protein complex.

In another aspect, the invention provides pharmaceutical compositions of inhibitory agents. More specifically, inventive pharmaceutical compositions comprise an effective amount of at least one inhibitory agent of the invention, or a physiologically tolerable salt thereof, and at least one pharmaceutically acceptable carrier.

In another aspect, the present invention provides methods for reducing or inhibiting viral DNA replication in a system by contacting the system with an effective amount of an inventive inhibitory agent. In certain embodiments, the viral DNA replication that is reduced or inhibited is that of HPV, and the inventive inhibitory agent that is used to contact the system inhibits HPV by inhibiting the E1-E2 protein-protein complex formation. Preferably, inhibitory agents used in these methods are small molecules. Examples of small molecules that can be used in the inventive methods are compounds 1, 2 and 3, whose chemical structures are presented in FIG. 1.

In another aspect, the present invention provides methods for treating a disease or medical condition associated with a papillomavirus. The inventive methods comprise administering to an individual in need thereof an effective amount of an inhibitory agent of the invention. In certain preferred embodiments, the virus is HPV and the inventive inhibitory agent that is administered to the patient inhibits HPV by inhibiting the E1-E2 protein-protein complex formation. Preferably, the inhibitory agent is a small molecule, such as compound 1, compound 2 or compound 3. The patient may be infected by a low-risk HPV or a high-risk HPV. The high-risk HPV may be HPV-16, HPV-18, HPV-31 or HPV-33. Preferably, the high-risk HPV is HPV-16.

The methods of treatment described herein may be used for inhibiting pathological progression of human papillomavirus infection, such as preventing or reversing the formation of warts (e.g., Plantar warts (verruca plantaris), common warts (verrucae vulgaris), Butcher's warts, flat warts, genital warts (condylomata acuminata), or epidermodysplasia verruciformis); as well as treating human papillomavirus lesions that have become, or are at risk of becoming, transformed and/or immortalized, i.e., cancerous (e.g., laryngeal papilloma, focal epithelial, cervical carcinoma). The inventive methods may also be used serially or in combination with chemotherapy, radiation, surgery, or other therapies with the goal of eliminating residual infected or pre-cancerous cells.

Other aspects, features and advantages of the present invention will become apparent from the following detailed description. It should be understood, however, that the detailed description and specific examples, while indicating preferred embodiments of the invention, are given by way of illustration only.

### BRIEF DESCRIPTION OF THE DRAWING

FIG. 1 shows the chemical structures of compounds 1, 2, and 3.

FIGS. 2A and 2B show results obtained by surface plasmon resonance for the binding of compound 2 (FIG. 2A) and compound 3 (FIG. 2B) to HPV-16 E2 protein.

### DEFINITIONS

Throughout the specification, several terms are employed, that are defined in the following paragraphs.

In the context of the present invention, the term “viral interacting protein” refers to any protein of viral origin that binds to a partner agent in such a way that the binding interaction is important or essential in the viral life cycle. Preferably, the binding interaction between the viral interacting protein and its partner is important or essential in the life cycle of a papillomavirus. In certain embodiments of the present invention, the binding interaction between the viral interacting protein and its partner is important or essential in the life cycle of HPV. Preferably, the viral interacting protein is the E1 or E2 protein. More preferably, the viral interacting protein is the HPV E2 protein.

As used herein, the terms “E1 protein” and “E2 protein” refer to those papillomavirus proteins that are encoded by the E1 and E2 open reading frames (ORFs) and that form a complex, which binds to papillomavirus DNA. The E1 and E2 proteins are known to play an important role in initiating viral DNA synthesis. The general structures of the papillomavirus E1 and E2 proteins are well known (see, for example, F. J. Hughes and M. A. Romanos, Nucleic Acids Res. 1993, 21: 5817-5823; P. J. Masterson et al., J. Virol. 1998, 72: 7407-7419; A. A. McBride et al., J. Biol. Chem. 1991, 266: 18411-18414; and I. Giri et al., EMBO J. 1988, 7: 2823-2829, which are incorporated herein by reference in their entirety). The E1 proteins encoded by the various papillomaviruses are well conserved and bear significant homology to the large T antigen replication protein of SV40 (P. Clertant and I. Seif, Nature, 1998, 311: 276-279; K. C. Mansky et al., J. Virol. 1997, 71: 7600-7608). The papillomavirus E1 protein is a nuclear phosphoprotein and ATP-dependent DNA helicase that binds to the origin of DNA replication, thus initiating viral plasmid replication. The minimal DNA binding domain of the E1 protein is found in the amino-terminus, while the carboxy-terminal region contains a domain that is necessary and sufficient for interaction with the E2 protein (V. G. Wilson et al., Virus Genes, 2002, 24: 275-290, which is incorporated herein by reference in its entirety). The papillomavirus E2 proteins are composed of two functional well-conserved domains connected by a hinge region (E. J. Androphy et al., Nature 1987, 325: 70-73; A. A. McBride et al., EMBO J. 1988, 7: 533-539; A. A. McBride et al., Proc. Natl. Acad. Sci. USA, 1989, 86: 510-514, which are incorporated herein by reference in their entirety). The E2 DNA binding and dimerization domain spans approximately 100 amino acids at the carboxy-terminal end, while the E2 amino-terminal region of approximately 200 amino acids features a transcriptional activation domain that is responsible for the regulation of viral gene expression and for interactions with components of the host cell apparatus. The E2 amino-terminal region also encompasses a domain which is critical for E1 interactions and viral DNA replication (R. S. Heldge, Annu. Rev. Biophys. Biomol. Struct. 2002, 31: 343-360, which is incorporated herein by reference in its entirety). In particular, the wild-type HPV-16 E1 protein is 649 amino acids long and has a molecular weight of 68 kDa, and the wild-type HPV-16 E2 protein is 365 amino acids long and has a molecular weight of 38 kDa. Within the amino-terminal region (amino acids 1 to 190) of HPV-16 E2 protein, a glutamic acid residue at position 39 (E39) has been identified as critical for interaction with E1 (H. Sakai et al., J. Virol. 1996, 70: 1602-1611; and H. Kasukawa et al., J. Virol. 1998, 72: 8166-8173, which are incorporated herein by reference in their entirety).

The terms “peptide” and “polypeptide” are used herein interchangeably. They refer to sequences of more than three amino acids. Preferably, peptides are sequences of less than about 250 amino acids, particularly, less than 100, 75, 50, and 30 amino acids. Preferred peptides of the invention contain in the range of about 5-50, 7-30, or 15-25 amino acids.

As used herein, the term “amino acid” refers to a monomeric unit of a protein. There are twenty amino acids found in naturally occurring proteins, all of which are L-isomers. The term “amino acid” may also include analogs of the amino acids, D-isomers of the protein amino acids and their analogs.

An inventive “interacting peptide” is a peptide that comprises a portion of a viral interacting protein that is sufficient to allow the peptide to bind to the partner of the interacting protein. Furthermore, the interacting peptide should include sequences that can be altered or mutated in such a way that the alteration or mutation reduces or destroys the ability of the peptide to bind the interacting protein's partner. In those embodiments of the invention that relate to HPV-16, preferred interacting peptides comprise a portion of the E2 protein that includes at least a region spanning the glutamic acid residue at position 39.

The term “wild-type” has its art understood meaning and refers to the naturally-occurring (or native) sequence of a protein or nucleic acid molecule.

The term “mutant” refers to a version of a protein or nucleic acid molecule that differs at a precise location from a wild-type version of the protein or nucleic acid. Differences may include deletions, substitutions, additions, and/or alterations. A mutant can differ at more than one precise location, however as will be appreciated by those of ordinary skill in the art, the overall sequence similarity to the wild-type is usually maintained.

As described herein, inventive “specificity peptides” are mutants of corresponding interacting peptides. The specificity peptide in a screening system is identical in amino acid sequence to the interacting peptide of the same screening system except that it contains one or more alteration or mutation that reduces or destroys its ability to bind to the interacting protein's partner. In certain embodiments of the invention, specificity peptides contain a single amino acid substitution or mutation. In those embodiments of the invention that relate to HPV-16, preferred specificity peptides are mutants of interacting peptides that comprise a portion of the native E2 protein including at least a region spanning the glutamic acid residue at position 39. More specifically, preferred specificity peptides are identical in amino acid sequence to corresponding interacting peptides, except that the glutamic acid residue at position 39 is substituted or mutated.

The term “isolated” when applied to interacting and specificity peptides of the present invention means a peptide or a portion thereof, which, by virtue of its origin or manipulation, (a) is present in a host cell as the expression product of a portion of an expression vector; (b) is linked to a protein or chemical moiety other than that to which it is linked in nature; (c) does not occur in nature, or (d) is such that its manufacture or production involved the hand of man. By “isolated” it is, alternatively or additionally, meant that the peptide of interest is chemically synthesized; or expressed in a host cell and purified away from at least some other proteins. Preferably, the peptide is also separated from substances such as antibodies or gel matrices (polyacrylamide) which are used to purify it.

The terms “inhibitor” and “inhibitory agent” are used herein interchangeably. They refer to a compound or factor that has been identified as capable of inhibiting protein-protein binding interactions which are important or essential to the life cycle of a virus. Preferred inhibitory agents of the invention inhibit the life cycle of a papillomavirus, such as HPV. For example, inhibitory agents inhibit the life cycle of HPV by inhibiting (e.g., precluding, reversing or disrupting) the E1-E2 protein-protein interaction. Preferably, inhibitory agents are small molecules.

The term “small molecule”, as used herein, refers to any natural or synthetic organic compound or factor with a molecular weight less than about 600-700 Daltons. Certain preferred small molecules are non-polymeric compounds.

The terms “fluorophore”, “fluorescent moiety”, and “fluorescent dye” are used herein interchangeably. They refer to a molecule which, in solution and upon excitation with light of appropriate wavelength, emits light back. The term “fluorescent labeling moiety” refers to a fluorescent molecule that can be covalently attached to a biomolecule (e.g., a protein or polypeptide) such that this biomolecule becomes detectable. Numerous known fluorescent labeling moieties of a wide variety of structures and characteristics are suitable for use in the practice of this invention. Similarly, methods and materials are known for covalently linking fluorophores to biomolecules such as polypeptides (see, for example, R. P. Haugland, “Molecular Probes: Handbook of Fluorescent Probes and Research Chemicals 1992-1994”, 5^{th} Ed., 1994, Molecular Probes, Inc.). Preferred fluorophores are photostable (i.e., they do not undergo significant degradation upon light excitation within the time necessary to perform the detection). Suitable fluorophores include, but are not limited to, fluorescein, rhodamine, cyanine, carbocyanine, allophycocyanine, phycoerythrin, umbelliferone, and their derivatives, analogues and combinations.

As used herein, the term “papillomavirus disease” refers to any kind of infection or disorder caused by a papillomavirus, including cancers and warts. Thus, the term includes symptoms and side effects of any papillomavirus infection, including latent, persistent and sub-clinical infections, whether or not the infection is clinically apparent.

As used herein, the term “treatment” refers to administration of an anti-viral agent to a patient (who is either the host of a papillomavirus infection or may be at risk of being infected by a papillomavirus). If it is administered prior to exposure to the virus, the treatment is preventive or prophylactic (i.e., it protects the patient against infection), whereas if the administration is performed after infection or initiation of the disease, the treatment is therapeutic (i.e., it combats the existing infection or cancer).

The term “individual” refers to a human or another mammal that can be the host of a papillomavirus, but may or may not be infected by the virus.

As used herein, the term “system” refers to a biological entity that can be the host of a papillomavirus. In the context of this invention, in vitro, in vivo, and ex vivo systems are considered. For example, the system may be a cell, a biological fluid, a biological tissue, or an animal. A system may, for example, originate from a live patient (e.g., it may be obtained by biopsy), or from a deceased patient (e.g., it may be obtained at autopsy).

As used herein, the term “biological fluid” refers to a fluid produced by and obtained from an individual. Examples of biological fluids include, but are not limited to, cerebrospinal fluid (CSF), blood serum, urine, and plasma. In the present invention, biological fluids include whole or any fraction of such fluids derived by purification, for example, by ultrafiltration or chromatography.

As used herein, the term “biological tissue” refers to a tissue obtained from an individual. The biological tissue may be whole or part of any organ or system in the body (e.g., skin, brain, pancreas, heart, kidney, gastrointestinal tract, thyroid gland, nervous system, eye, skin, and the like).

A “pharmaceutical composition”, as used herein, is defined as comprising an effective amount of at least one inhibitory agent of the invention, or a physiologically tolerable salt thereof, and at least one pharmaceutically acceptable carrier.

As used herein, the term “effective amount” refers to any amount of an inhibitory agent, or pharmaceutical composition thereof, that is sufficient to fulfill its intended purpose(s). For example, the purpose(s) may be: to protect against infection by a papillomavirus; to combat a papillomavirus; to prevent the onset of a disease caused by the virus; to slow down or stop the progression, aggravation, or deterioration of the symptoms of a papillomavirus disease; to bring about amelioration of the symptoms of the disease; or to cure the disease.

The term “physiologically tolerable salt” refers to any acid addition or base addition salt that retains the biological activity and properties of the corresponding free base or free acid, respectively, and that is not biologically or otherwise undesirable. Acid addition salts are formed with inorganic acids (e.g., hydrochloric, hydrobromic, sulfuric, nitric, phosphoric acids, and the like); and organic acids (e.g., acetic, propionic, pyruvic, maleic, malonic, succinic, fumaric, tartaric, citric, benzoic, mandelic, methanesulfonic, ethanesulfonic, p-toluenesulfonic, salicylic acids, and the like). Base addition salts can be formed with inorganic bases (e.g., sodium, potassium, lithium, ammonium, calcium, magnesium, zinc, aluminum salts, and the like) and organic bases (e.g., salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethyl-aminoethanol, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like).

As used herein, the term “pharmaceutically acceptable carrier” refers to a carrier medium which does not interfere with the effectiveness of the biological activity of the active ingredients and which is not excessively toxic to the host at the concentrations at which it is administered. The term includes solvents, dispersion media, coatings, antibacterial and antifungal agents, isotonic agents, absorption delaying agents, and the like. The use of such media and agents for pharmaceutically active substances is well known in the art (see, for example, “Remington's Pharmaceutical Sciences”, E. W. Martin, 18^{th} Ed., 1990, Mack Publishing Co.: Easton, Pa., which is incorporated herein by reference in its entirety).

Additional definitions are provided throughout the Detailed Description.

### DETAILED DESCRIPTION OF CERTAIN PREFERRED EMBODIMENTS

The present invention relates to the identification, production and/or use of anti-viral agents. In particular, the invention relates to the identification, production and/or use of anti-viral agents that inhibit or disrupt protein-protein interactions that are important or essential in the viral life cycle.

I. Screening Systems

In one aspect, the invention provides screening systems including an interacting peptide and a specificity peptide.

Inventive Peptides

An inventive interacting peptide is a peptide which comprises a portion of a viral interacting protein that is sufficient to allow the peptide to bind to the partner of the interacting protein. The corresponding inventive specificity peptide comprises an amino acid sequence identical to that of the interacting peptide except that it has an alteration or a mutation that reduces or destroys its ability to bind to the viral interacting protein's partner. Therefore, the interacting peptide should include sequences that can be altered or mutated in such a way that the alteration or mutation reduces or destroys the ability of the peptide to bind the viral interacting protein's partner.

Generally, a suitable portion of a viral interacting protein to be included in an inventive interacting peptide is one that, when produced as a peptide, can compete with the native protein-protein interaction. Preferably, the portion retains more than between about 50 and 80% of the binding affinity of the native viral interacting protein. More preferably, the portion retains more than about 80% of the binding affinity of the native viral interacting protein. Most preferably, the portion has an identical or higher binding affinity than the viral interacting protein.

Useful portions of a viral interacting protein (i.e., fragments that are sufficient to allow binding to the interacting protein's partner) may be identified by any suitable method known in the art. For example, identification of useful portions may be carried out by testing several different fragments of the viral interacting protein for their ability to bind to the interacting protein's partner. Other suitable ways of identifying useful portions include directed mutagenesis methods such as alanine substitution mutagenesis (B. C. Cunningham et al., Science, 1989, 244: 1081-1095). In this method, each residue of a specific fragment (or portion) of a viral interacting protein is systematically substituted with alanine and the mutant peptides generated by alanine substitutions are then tested for their ability to compete with the binding interaction between the viral interacting protein and its partner.

Such a structure-function analysis carried out in the Applicants' laboratory led to the identification of the glutamic acid residue at position 39 on the HPV-16 E2 protein as essential for the formation of the E1-E2 protein-protein complex and for its function as an auxilliary replication factor (H. Sakai et al., J. Virol. 1996, 70: 1602-1611, which is incorporated herein by reference in its entirety). Interestingly, the glutamic acid residue at position 39 is conserved among many E2 proteins in various bovine and human papillomavirus strains (e.g., BPV-1, HPV-6b, HPV-11, HPV-31, HPV-1A and HPV-57), including several high risk strains (e.g., HPV-16 and HPV-18). Furthermore, the authors of this work later showed that 15- and 23-mer peptides corresponding to a region of the HPV-16 E2 protein flanking the glutamic acid residue at position 39 were capable of preventing the E1 and E2 proteins from binding, and most importantly could inhibit papillomavirus DNA replication in vitro (H. Kasukawa et al., J. Virol. 1998, 72: 8166-8173, which is incorporated herein by reference in its entirety). The efficacy of these peptides was not exclusive to HPV-16: they were also found to inhibit interactions of HPV-11 E1 with the E2 proteins of both HPV-11 and HPV-16, and to inhibit in vitro replication with the same combinations of E1 and E2 proteins.

In certain embodiments, the binding interaction between the viral interacting protein and its partner is important or essential to the life cycle of a papillomavirus. In other embodiments, interactions between the viral interacting protein and its partner are important or essential to the life cycle of HPV. Preferably, in the methods of the invention the viral interacting protein is the E1 protein or the E2 protein.

The general structures of the papillomavirus E1 and E2 proteins are well known (see, for example, F. J. Hughes and M. A. Romanos, Nucleic Acids Res. 1993, 21: 5817-5823; P. J. Masterson et al., J. Virol. 1998, 72: 7407-7419; A. A. McBride et al., J. Biol. Chem. 1991, 266: 18411-18414; and I. Giri et al., EMBO J. 1988, 7: 2823-2829, which are incorporated herein by reference in their entirety). The papillomavirus E1 protein is the most conserved papillomavirus protein; it is a nuclear phosphoprotein and ATP-dependent DNA helicase that binds to the origin of DNA replication, thus initiating viral plasmid replication. The minimal DNA binding domain of the E1 protein is found in the amino-terminus, while the carboxy-terminal region contains a domain that is necessary and sufficient for interaction with the E2 protein (V. G. Wilson et al., Virus Genes, 2002, 24: 275-290, which is incorporated herein by reference in its entirety). E2 proteins are composed of two well-conserved functional domains connected by a hinge region. The E2 DNA binding and dimerization domain spans approximately 100 amino acids at the carboxy-terminal end. The E2 amino-terminal region of approximately 200 amino acids features a transcriptional activation domain that is responsible for the regulation of viral gene expression and for interactions with components of the host cell apparatus, and encompasses a domain which is critical for E1 interactions and viral DNA replication (R. S. Heldge, Annu. Rev. Biophys. Biomol. Struct. 2002, 31: 343-360, which is incorporated herein by reference in its entirety).

Preferably, the viral interacting protein is a HPV E2 protein. More preferably, the viral interacting protein is the HPV-16 E2 protein. In certain embodiments of the invention, interacting peptides correspond to a portion of the HPV-16 E2 protein including at least a region spanning the glutamic acid residue at position 39 (E39); and specificity peptides are mutants of these interacting peptides that contain an alteration or mutation at E39. Specificity peptides may contain more than one alteration or mutation, however, the overall sequence similarity to the corresponding interacting peptides should be maintained. For example, in preferred specific peptides of the invention E39 may be substituted by an alanine residue. However, such a mutation need not be an alanine substitution, but may, alternatively, be any similar substitution or alteration that reduces or destroys the ability of the resulting specificity peptide to bind to the viral interacting protein's partner. Preferably, the specificity peptide exhibits less than 10% of the binding affinity of the interacting peptide. More preferably, the specificity peptide exhibits less than 5% of the binding affinity of the interacting peptide. Most preferably, the specificity peptide does not bind to the viral interacting protein's partner.

An example of a screening system is one that comprises a 23-mer interacting peptide corresponding to a region (amino acids 28 to 50) of the full-length, native HPV-16 E2 protein flanking the E39 residue which has the following amino acid sequence:

A-H-I-D-Y-W-K-H-M-R-L-E-C-A-I-Y-Y-K-A-R-E-M-G,

and a 23-mer specificity peptide, whose amino acid sequence:

A-H-I-D-Y-W-K-H-M-R-L-A-C-A-I-Y-Y-K-A-R-E-M-G,

is identical to that of the interacting peptide except that the glutamic acid residue (at position 39 in the native, full length E2 protein) has been mutated to an alanine residue. These interacting and specificity peptides and their use as a screening system are described in Example 1.
Peptide Preparation

The isolated peptides in the screening systems of the invention may be prepared by any suitable method known in the art. For example, the peptides may be obtained by chemical synthesis or by recombinant methods.

The peptides of the invention are generally sufficiently short that chemical synthesis, using standard methods is feasible. Solid-phase peptide synthesis, which was initially described by R. B. Merrifield (J. Am. Chem. Soc. 1963, 85: 2149-2154) is a quick and easy approach to synthesizing peptides and small peptidic molecules of known sequences. A compilation of such solid-phase techniques may be found, for example, in “Solid Phase Peptide Synthesis” (Methods in Enzymology, G. B. Fields (Ed.), 1997, Academic Press: San Diego, Calif., which is incorporated herein by reference in its entirety). Most of these synthetic procedures involve the sequential addition of one or more amino acid residues or suitably protected amino acid residues to a growing peptide chain. For example, the carboxy group of the first amino acid is attached to a solid support via a labile bond, and reacted with the second amino acid, whose amino group has, beforehand, been chemically protected to avoid self-condensation. After coupling, the amino group is deprotected (i.e., the protecting group is chemically removed), and the process is repeated with the following amino acid. Once the desired peptide is assembled, it is cleaved off from the solid support, precipitated, and the resulting free peptide may be analyzed and/or purified as desired. Solution methods, as described, for example, in “The Proteins” (Vol. 11, 3^{rd} Ed., H. Neurath et al. (Eds.), 1976, Academic Press: New York, N.Y., pp. 105-237) may also be used to synthesize the peptides of the invention.

Alternatively, the peptides of the screening systems provided herein can be produced by recombinant DNA methods. These methods generally involve isolation of the gene encoding the desired protein, transfer of the gene into a suitable vector, and bulk expression in a cell culture system. The DNA coding sequences for the polypeptides of the invention are sufficiently short to be readily prepared synthetically using methods known in the art (see, for example, M. P. Edge et al., Nature, 1981, 292: 756-762).

After synthesis, the DNA encoding the desired peptide is inserted into a recombinant expression vector. A recombinant expression vector may be a plasmid, phage, viral particle, or other nucleic acid molecule containing vectors or nucleic acid molecule containing vehicles which, when introduced into an appropriate host cell, contains the necessary genetic elements to direct expression of the coding sequence of interest. Standard techniques well known in the art can be used to insert the nucleic acid molecule into the expression vector. The insertion results in the coding sequence being operatively linked to the necessary regulatory sequences (i.e., the expression control sequence and encoding sequence are arranged in three-dimensional space relative to one another such that the control sequence directs, modifies, or otherwise effects the expression of the encoding sequence).

Host cells for use in the production of proteins are well known and readily available. Examples of host cells include bacteria cells such as Escherichia coli, Bacillus subtilis, attenuated strains of Salmonella typhimurium, and the like; yeast cells such as Saccharomyces cerevisiae, Schizosaccharomyces pombe, Kluyveromyces strains, Candida, or any yeast strain capable of expressing heterologous proteins; insect cells such as Spodoptera frugiperda; non-human mammalian tissue culture cells such as Chinese Hamster Ovary (CHO) cells, monkey COS cells, and mouse 3T3 cells; and human tissue culture cells such as HeLa cells, HL-60 cells, kidney 293 cells and epidermal S431 cells.

Several expression vectors to produce polypeptides in well known expression systems are conveniently commercially available. For example, the plasmids pSE420 (available from Invitrogen, San Diego, Calif.) and pBR322 (available from New England Biolabs, Beverly, Mass.) may be used for the production of the inventive peptides in E. coli. Similarly, the plasmid pYES2 (Invitrogen) may be used for peptide production in S. cerevisiae strains of yeast. The commercially available MacBacR™ kit (Invitrogen) for baculovirus expression system or the BaculoGold™ Transfection Kit available from PharMingen (San Diego, Calif.) may be used for production in insect cells, while the plasmids pcDNA I, pcDNA 3, and pRc/RSV, commercially available from Invitrogen, may be used for the production of the peptides of the invention in mammalian cells such as Chinese Hamster Ovary (CHO) cells.

Other expression vectors and systems can be obtained or produced using methods well known to those skilled in the art. Expression systems containing the requisite control sequences, such as promoters and polyadenylation signals, and preferably enhancers are readily available for a variety of hosts (see, for example, Sambrook et al., Molecular Cloning: A Laboratory Manual, 2^{nd} Ed., 1989, Cold Spring Harbor Press: Cold Spring, N.Y.; and R. Kaufman, Methods in Enzymology, 1990, 185: 537-566).

The expression vector including the DNA that encodes the desired peptide of the invention is used to transform the compatible host cell. The host cell is then cultured and maintained under conditions favoring expression of the desired peptide. The peptide thus produced is recovered and isolated, either directly from the culture medium or by lysis of the cells. It can then be characterized by different methods such as Nuclear Magnetic resonance (NMR) and X-ray crystallography.

II. Identification of Anti-Viral Agents

In another aspect, the present invention provides a system for identifying anti-viral agents. This system allows candidate compounds or factors to be contacted with the interacting and specificity peptides followed by detection of the relative binding. Those compounds or factors that bind to the interacting peptide to a greater extent than to the specificity peptide are classified as inhibitory agents.

Preferably, detection of the binding is carried out quantitatively. Preferred compounds or factors that are classified as inhibitory agents bind to the interacting peptide about 100 to 1000 times more efficiently than to the specificity peptide. Preferably, compounds or factors that are classified as inhibitory agents bind to the interacting peptide about 500 to 1000 times more efficiently than to the specificity peptide. More preferably, inhibitory agents bind to the interacting peptide but do not bind significantly (i.e., to a detectable extent) to the specificity peptide.

Detection of the Binding

Detection of the relative binding may be carried out by any of a variety of methods. For example, the relative binding can be determined by using interacting and specificity peptides that are labeled with detectable agents. Suitable detectable agents for use in the present invention include, but are not limited to, optical, radioactive and fluorescent moieties. Preferably, the label is selected such that it results in a signal which can be measured and is related (e.g., proportional) to the amount of label in the sample.

Labeling methods are well known in the art. The most convenient and widely used chemical function for protein and peptide labeling is the primary amino group provided by the ε-amine of lysine or by the amino-terminus. In most cases, one or more lysine residues will be accessible to labeling reagents. The most useful reaction for labeling at amino groups is acylation. This can be achieved by activating the labeling molecule in situ with activating agents such as carbodiimide or by using stable, active ester derivatives of the labeling molecule, in particular, N-hydroxysuccinimide(NHS)-esters. Labeling of proteins or peptides is generally performed in aqueous buffer. Hydrolysis of the NHS-ester by water is a major competing process of the acylation reaction; hydrolysis increases with increasing pH and with decreasing protein concentration in solution. However, most protein labeling reactions are efficiently carried out at pH values between 7 and 9 using phosphate, bicarbonate/carbonate, or borate buffers, or any other buffers that do not contain a source of primary or secondary amines (e.g., Tris).

The interacting and specificity peptides of the invention may be labeled with a radioisotope such as ³H, ³²P, ³⁵S, ¹⁴C, ¹²⁵I, and the like, using well-known methods (see, for example, D. S. Wilbur, Bioconj. Chem. 1992, 3: 433-470; and U.S. Pat. Nos. 3,979,506 and 5,045,303). The binding assay may be performed by, for example, immobilizing a candidate compound or factor on a support, contacting it with the radiolabeled interacting peptide, and washing out the unreacted polypeptide. The radioactivity of the bound interacting peptide can then be detected using standard techniques. The same experiment may then be carried out using the specificity peptide, and the radioactivity of the bound specificity peptide is similarly detected. Comparison of the radioactive signals measured for the interacting and specificity peptides allows determination of the relative binding.

As will be appreciated by those skilled in the art, it may be desirable to employ a nonradioactive signal to detect the relative binding, such as optical density (or color intensity) or fluorescence.

Preferably, the interacting and specificity peptides of the invention are labeled with a fluorescent moiety, such as fluorescein, rhodamine, cyanine, carbocyanine, phycoerythrin, umbelliferone, Texas red, fluorescein isothiocyanate (FITC), merocyanine, styryl dye, BODIPY dye, Cy-3™ or Cy-5™ (i.e., 3- or 5-N,N′-diethyltetramethylindodicarbocyanine) and the like. Methods to label proteins and peptides with fluorescent dyes are well known in the art (see, for example, R. P. Haugland, “Molecular Probes: Handbook of Fluorescent Probes and Research Chemicals 1992-1994”, 5^{th} Ed., 1994, Molecular Probes, Inc). Such fluorescent dyes are commercially available as NHS-esters, maleimides, and hydrazides to make them suitable for labeling via reaction with amine, thiol and aldehyde groups, respectively. Fluorescent labeling dyes as well as labeling kits are commercially available from, for example, Amersham Biosciences Inc. (Piscataway, N.J.), Molecular Probes Inc. (Eugene, Oreg.), and New England Biolabs Inc. (Berverly, Mass.).

Favorable properties of fluorescent labeling moieties to be used in the practice of the invention include high molar absorption coefficient, high fluorescence quantum yield, and photostability. Preferred labeling fluorophores exhibit absorption and emission wavelengths in the visible (i.e., between 400 and 750 nm) rather than in the ultraviolet range (i.e., below 400 nm) of the spectrum to avoid possible interference from the small molecule(s) to be screened.

In one embodiment of the invention, the binding assay is carried out according to a method developed by certain of the present inventors (G. MacBeath et al., J. Am. Chem. Soc. 1999, 121: 7967-7968, which is incorporated herein by reference in its entirety). Accordingly, a candidate compound or factor to be tested is immobilized on a support and contacted with a fluorescently labeled interacting peptide. This is followed by several washes to remove any unreacted polypeptide. The fluorescence emitted by the bound interacting peptide can then be detected by standard steady-state fluorimetry methods. The same experiment is then carried out using the specificity peptide, and the fluorescence emitted by the bound specificity peptide is detected. Comparison of the fluorescence signals measured for the interacting and specificity peptides allows determination of the relative binding.

For example, interacting and specificity peptides of the invention may be labeled with the fluorescent dye, Cy5. This carbocyanine fluorophore exhibits a maximum absorption at 675 nm; emits fluorescence with a maximum at 694 nm; and its fluorescence quantum yield has been determined to be 0.23 when it is conjugated to a protein (Amersham Biosciences Inc., Piscataway, N.J.). 23-mer HPV-16 E2-derived interacting and specificity peptides labeled with Cy5 are described and used in Example 1.

The inventive methods may also be carried out using interacting and specificity peptides labeled with a lanthanide chelate (such as, for example, Europium-cryptate). Upon excitation, lanthanide chelates give an intense and long-lived fluorescence emission (>500 μs). This long-lived emission can be detected significantly after excitation, which eliminates the short-lived (i.e., 10-100 ns) background fluorescence emitted by any other organic fluorophore present during the assay. Lanthanide chelates also display a large Stokes' shift (i.e., the difference between the wavelength of maximum absorption and the wavelength of maximum emission) compared with traditional fluorescent labels (i.e., Stokes' shift>300 nm instead of a few nanometers), which minimizes crosstalk between excitation and emission signals and contributes to a high signal to noise ratio.

In such lanthanide-based methods, a candidate compound or factor to be tested is immobilized on a support and contacted with an interacting peptide labeled with a lanthanide chelate. After washing to remove any unreacted polypeptide, the fluorescence emitted by the bound interacting peptide is detected by standard time-resolved fluorescence methods. The same experiment is then carried out using the specificity peptide labeled with a lanthanide chelate. Comparison of the amplitudes of the fluorescence signals measured for the interacting and specificity peptides allows determination of the relative binding.

Labeling of biomolecules with lanthanide chelates is well known in the art (see, for example, I. Hemmila et al., Anal. Biochem. 1984, 137: 335-343; E. P. Diamandis and R. C. Morton, J. Immunol. Methods, 1988, 112: 43-52; and A. R. Holzwarth, Methods of Enzymology, 1995; 246: 334-362). Lanthanide chelates are commercially available from, for example, Amersham Biosciences Inc. (Piscataway, N.J.).

II. Candidate Compounds or Factors

In certain embodiments, the inventive methods are used for identifying compounds or factors that are capable of inhibiting or disrupting protein-protein interactions that are important in the life cycle of a papillomavirus with the goal of developing anti-viral agents. Preferably, the papillomavirus is a human papillomavirus. More preferably, the inventive methods are used for identifying agents that inhibit HPV DNA replication by inhibiting (e.g., precluding, reversing, or disrupting) the formation of the E1-E2 protein-protein complex.

As will be appreciated by those of ordinary skill in the art, any kind of compounds or factors can be tested using the inventive screening methods. In certain embodiments, small molecules (i.e., any natural or synthetic organic compounds or factors with a molecular weight lower than about 600-700 Daltons) are tested using the inventive methods. In other embodiments, the inventive methods are used for screening small molecule collections or libraries. Any collection or library of small molecules can be screened using the methods provided herein. As used herein, the term “collection” refers to any set of compounds, molecules, or agents, while the term “library” refers to any set of structural analogs of compounds, molecules or agents.

Traditional approaches to the identification and characterization of new and useful drug candidates include the generation of large collections or libraries of compounds followed by testing against either known or unknown targets (see, for example, WO 94/24314; WO 95/12608; M. A. Gallop et al., J. Med. Chem. 1994, 37: 1233-1251; E. M. Gordon et al., J. Med. Chem. 1994, 37: 1385-1401). Both natural product and chemical compounds may be tested b the methods of the invention.

Natural product collections are generally derived from microorganisms, animals, plants, or marine organisms; they include polyketides, non-ribosomal peptides, and/or variants (non-naturally occurring) thereof (for a review, see, for example, D. E. Cane et al., Science, 1998, 82: 63-68). Chemical libraries often consist of structural analogs of known compounds or compounds that are identified as “hits” or “leads” via natural product screening. Chemical libraries are relatively easy to prepare by traditional automated synthesis, PCR, cloning or proprietary synthetic methods (for a review of combinatorial chemistry and libraries created therefrom, see, for example, P. L. Myers, Curr. Opin. Biotechnol. 1997, 8: 701-707).

In preferred embodiments, methods of the invention are used to screen small organic molecule collections or libraries. The development of solid-phase organic synthesis, which permits reactions to be automated and run in parallel, has allowed the generation of such small organic molecule libraries (L. A. Thompson and J. A. Ellman, Chem. Rev. 1996, 96: 555-600). Introduction of the split-pool synthetic method (A. Furka et al., Int. J. Pept. Protein Res. 1991, 37: 487-493), a variation of the solid-phase organic synthesis, which treats each solid-phase particle (commonly, derivatized polystyrene beads) as a separate reaction vessel, has increased the productivity by allowing the generation of millions of distinct compounds. By splitting and pooling the collection of synthesis beads over a reaction sequence, all possible combinations of a large matrix of reagents and building blocks can be accessed, which greatly amplifies the number of compounds produced for a small number of reactions performed (K. S. Lam et al., Nature, 1991, 354: 82-84; R. Houghten et al., Nature, 1991, 354: 84-86).

As illustrated by Example 1, the inventive methods can be used to screen such collections or libraries with the goal of identifying small molecule therapeutics to control HPV infection. In Example 1, the screening of a small molecule library of 1890 1,3-dioxanes, generated by split-pool synthesis in the Applicants' laboratory (S. M. Sternson et al., J. Am. Chem. Soc. 2001, 123: 1740-1747, which is incorporated herein by reference in its entirety) has led to the identification of compound 1, along with nine other molecules.

The screening of small molecule libraries according to the inventive methods provides “hits” or “leads”, i.e., compounds that possess a desired but not-optimized biological activity. The next step in the development of useful drug candidates is usually the analysis of the relationship between the chemical structure of a hit compound and its biological or pharmacological activity. Molecular structure and biological activity are correlated by observing the results of systemic structural modification on defined biological endpoints. For example, comparison of the affinity of structurally-related compounds helps identify positions on ligands that are important for binding to the target biomolecule; analysis of the effects of the stereochemistry of these compounds (i.e., the arrangement of their atoms in space) on their binding ability helps identify conformations that are favorable to ligand/target complex formation. Structure-activity relationship information available from the first round of screening can then be used to generate small secondary libraries which are subsequently screened for compounds with higher affinity. The process of performing synthetic modifications of a biologically active compound to fulfill all stereoelectronic, physicochemical, pharmacokinetic, and toxicologic factors required for clinical usefulness is called lead optimization.

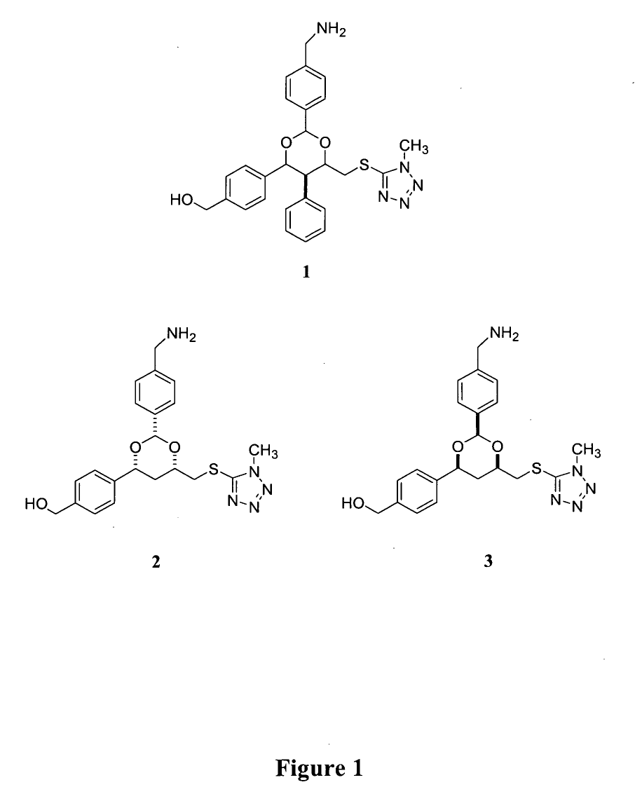

The small molecules identified by the screening methods of the invention can similarly be subjected to a structure-activity analysis, and chemically modified to provide improved drug candidates. Example 2 illustrates such chemical modifications. Compounds 2 and 3, the chemical structures of which are presented in FIG. 1, are derivatives of compound 1 that lack the unsubstituted phenyl group. These compounds have been synthesized with the goal of generating molecules with improved solubility properties. Compounds 2 and 3 are enantiomers of the same molecule (i.e., compounds that are optical isomers, or mirror images of one another).

III. Characterization of Anti-Viral Agents

In another aspect, the present invention relates to specific inhibitory agents identified by the inventive screening methods astrice capable of inhibiting or disrupting key protein-protein interactions that are important in the life cycle of a papillomavirus. Preferred inhibitory agents of the invention inhibit or disrupt key protein-protein interactions that are important in the life cycle of HPV. Preferably, inventive inhibitory agents inhibit HPV DNA replication by inhibiting (e.g., precluding, reversing, or disrupting) the formation of the E1-E2 protein-protein complex. In certain embodiments, the inhibitory agents are small molecules. Preferred inhibitory agents are small molecules that are cell permeable. The present invention also provides chemical derivatives of these small molecules, for example, those derivatives that are developed through lead optimization, as described above.

As will be appreciated by those skilled in the art, it is generally desirable to further characterize the inhibitory agents identified by the inventive screening methods. It may, for example, be desirable to evaluate their inhibitory activity with regard to the protein-protein interaction of interest. In those embodiments that relate to HPV, it may, for example, be desirable to evaluate the ability of inhibitory agents to bind to the native HPV E2 protein, to inhibit the E1-E2 protein-protein complex formation, and/or to inhibit HPV DNA replication. This characterization may be performed by using any of various available approaches. For example, surface plasmon resonance may be used to further evaluate the binding activity of inhibitory agents.

Surface plasmon resonance allows the observation of the association and dissociation of interacting molecules in real-time (G. Panayotou, Methods Mol. Biol. 1998, 88: 1-10). Briefly, surface plasmon resonance is based on an optical phenomenon that occurs in a thin metal film at an optical interface under conditions of total internal reflection. The system uses polarized light to detect subtle changes in optical resonance that occur from a target biomolecule immobilized on the thin metal film when it is contacted by test compounds in solution. When a test compound binds to the immobilized target biomolecule, the refractive index increases. When the test compound and biomolecule dissociate, the refractive index decreases. By allowing the variation of the refractive index to be monitored in real-time, surface plasmon resonance provides information on the binding kinetics, affinity and specificity. In particular, surface plasmon resonance can be used to characterize and analyze the binding of small molecules to therapeutic targets.

Example 3 illustrates the use of surface plasmon resonance to determine the equilibrium dissociation constants for the binding of compound 2 and compound 3 to the native HPV-16 E2 protein.

As will be appreciated by those of ordinary skill in the art, a variety of assays may be used to assess and evaluate the ability of inhibitory agents to prevent or disrupt key protein-protein interactions. The characterization may be performed using a competitive assay or an ELISA assay.

For example, inhibitory agents identified by the inventive methods as capable of binding to an E2-derived interacting peptide and not to the corresponding specificity peptide, may be tested for their ability to preclude, reverse or disrupt the E1-E2 protein-protein complex formation using an ELISA assay. In such an assay, the native E1 protein may be bound to a support via an antibody. The native E2 protein is then added under appropriate reaction conditions of pH, salt concentration, etc., in the presence or absence of an inhibitory agent evaluated for the development of HPV anti-viral agents. Preferred inhibitory agents inhibit or reduce E1-E2 complex formation compared to the controls. Controls may be molecules, compounds, or agents that are known to have no inhibitory effects on the formation of the E1-E2 complex. Preferably, the extent of complex formation is monitored using a labeled molecule that binds to the E2 protein. For example, the labeled molecule may be an antibody specific for E2.

The antibody used to bind the native E1 protein to a support may be immobilized on any appropriate solid support using any appropriate technique. Any combination of support and binding technique that leaves the antibody immunoreactive, yet sufficiently immobilizes the antibody so that it can be retained with any bound antigen during a washing, can be employed. The solid support may be any suitable insoluble carrier material for the binding of antibodies in immunoassays. Suitable materials are known in the art and include, but are not limited to, nitrocellulose sheets or filters, agarose, resin, plastic (e.g., PVC), latex, metal beads, and the like. Various methods of immobilizing antibodies on solid support are known in the art (see, for example, I. Silman and E. Katchalski, Ann. Rev. Biochem. 1966, 35: 873-908). Such methods include covalent coupling, direct adsorption, physical entrapment, and attachment to a protein-coated surface.

The antibody specific for the E2 protein can desirably be labeled in such a way that it allows for the detection of the antibody when bound to a support. Generally, the labelling directly or indirectly results in a signal which is measurable and related (e.g., proportional) to the amount of label present in the sample. For example, directly measurable labels include radio-labels (e.g., ¹²⁵I, ³⁵S, ¹⁴C; etc.). A preferred directly measurable label is an enzyme conjugated to the antibody, which produces a color reaction in the presence of the appropriate substrate (e.g., horseradish peroxidase/ortho-phenylenediamine). An indirectly measurable label is, for example, a biotinylated antibody. In this case, the detection is carried out by contacting the biotinylated antibody with a solution containing a labeled avidin complex, which results in the binding of the avidin to the biotinylated antibody. A preferred example of an indirect label is the avidin/biotin system employing an enzyme conjugated to avidin, the enzyme producing a color reaction.

It will often be preferred that a nonradioactive signal, such as optical density (or color intensity) produced by an enzyme reaction be employed for detection. Numerous enzyme/substrate combinations, which can produce a suitable signal, are known in the art (see for example, U.S. Pat. Nos. 4,323,647 and 4,190,496).

The anti-E2 antibody used in the ELISA assay described above can be polyclonal or monoclonal. As will be readily appreciated by those of ordinary skill in the art, the ELISA assay may, alternatively, be carried out by binding the native E2 protein to a solid support via an antibody and using a labeled antibody that binds to the E1 protein.

The inhibitory agents identified by the screening methods of the invention may be further tested in biological assays that allow for the determination of the inhibitor's properties in vitro, for example, in cell-based assays (i.e., cell culture systems) and/or in vivo, in animal models of HPV infection (vide infra).

The ability of inhibitory agents of the invention to reduce or inhibit viral DNA replication may be evaluated by any suitable method. In particular, a variety of assays are available to test the inhibitory activity with regard to the viral DNA replication. Cell-based assays that assess the antiviral activity of drug candidates against HPV are known in the art (see, for example, U.S. Pat. No. 5,541,058, or P. R. Clark et al., Antiviral Res. 1998, 37: 97-106, which describes a cell-based assay that allows the HPV DNA replication to be monitored in the presence of putative anti-viral agents). The anti-viral efficacy of the inhibitors of the invention can be determined by generating dose response curves from data obtained using various drug concentrations. The in vitro biological activity of the inhibitory agents of the invention can thus be evaluated, compared to one another as well as to known active compounds or clinically relevant compounds which can be used as positive controls.

One challenge in developing anti-viral agents effective against DNA viruses such as human papillomaviruses has been dependent on finding an animal model which mimics the human form of the disease. Since papillomavirus infections are species-specific, there is no exact animal model system for HPV infection. However, common mechanisms of regulation or transcription and replication exist among different species (I. Giri and M. Yaniv, EMBO J. 1988, 7: 2823-2829; C. M. Chiang et al., J. Virol. 1992, 66: 5224-5231; and C. M. Chiang et al., Proc. Natl. Acad. Sci. U.S.A. 1992, 89: 5799-5803) and animal papillomaviruses have been widely used as models to study papillomavirus infection in humans. In particular, many similarities have been identified between cottontail rabbit papillomavirus (CRPV) and HPV infections (X. Wu et al., J. Virol. 1994, 68: 6097-6102; J. L. Brandsma, Intervirol. 1994, 37: 189-200; J. W. Kreider et al., Adv. Cancer Res. 1981, 35: 81-100). This led to the development of a model based on domestic rabbits which efficiently grow cutaneous papillomas (warts) when infected with cottontail rabbit papillomavirus (CRPV). This animal model can desirably be used to evaluate the ability of inventive inhibitors to reduce or inhibit the growth of these cutaneous lesions as well as to assess parameters such as cytotoxicicy, bioavailability, and pharmacokinetics.

Inhibitory agents of the invention may also be tested in ex vivo assays. Several ex vivo cervical carcinoma models have been developed (see, for example, S. E. Waggoner et al., Gynecol. Oncol. 1990, 38: 407-412.; W. Bonnez et al., Virology, 1993, 197: 455-458; W. Bonnez et al., J. Virol. 1998, 72: 5256-5261; and K. S. Tewari et al., Gynecol Oncol. 2000, 77: 137-148). These models are all based on Kreider's model in which primary keratinocytes, or human cervical or foreskin tissue is exposed to HPV in vitro and then grafted beneath the renal capsule of an immunocompromised mouse (M. K. Howett et al., Intervirol. 1990, 31: 109-115). Inhibitory agents of the invention can be tested in these ex vivo systems to determine their ability to reduce or inhibit the HPV proliferation in cervical carcinoma models.

IV. Inhibition of Viral Life Cycle

The present invention also provides methods for reducing or inhibiting viral replication in a system by contacting the system with an effective amount of an inventive inhibitory agent. In particular, these methods allow for the reduction or inhibition of DNA replication of papillomaviruses. In certain embodiments, the viral replication that is reduced or inhibited is that of HPV, and the inventive inhibitory agent that is used to contact the system inhibits HPV DNA replication by inhibiting (e.g., precluding, reversing, or disrupting) the E1-E2 protein-protein complex formation. Preferably, the inhibitory agents used in these methods are small molecules, such as, for example, compound 1, compound 2 or compound 3.

The contacting can be carried out in vitro, in vivo, or ex vivo. The method can be performed in cell-free or cell-based systems, in biological samples such as biological fluids and excised tissues, or in animal models. A cell, biological fluid, or biological tissue may, for example, originate from a patient infected or suspected to be infected by a papillomavirus. These biological samples may originate from a live patient (e.g., they may be obtained by biopsy), or from a diseased patient (e.g., they may be obtained at autopsy).

Preferably, the papillomavirus is HPV. In certain embodiments, the human papillomavirus is a low risk HPV. In other embodiments, the human papillomavirus is a high-risk HPV. Preferably, the high-risk HPV is selected from the group consisting of HPV-16, HPV-18, HPV-31, and HPV-33. More preferably, the high-risk HPV is HPV-16.

V. Methods of Treatment

The present invention is also directed to methods for treating a disease or medical condition associated with a papillomavirus virus. Preferably, the virus is a human papillomavirus. The inventive methods comprise administering to an individual in need thereof an effective amount of an inhibitory agent of the invention. As used herein, the term “effective amount” refers to any amount of inhibitory agent that is sufficient to inhibit or lessen the spread of HPV infection, to reduce the symptoms of the specific HPV-associated disease, or to prevent their recurrence.

In preferred embodiments, the method is used for inhibiting pathological progression of HPV infection, such as preventing or reversing the formation of warts (e.g., Plantar warts (verruca plantaris), common warts (verrucae vulgaris), Butcher's warts, flat warts, genital warts (condylomata acuminata), or epidermodysplasia verruciformis); as well as for treating human papillomavirus lesions which have become, or are at risk of becoming, transformed and/or immortalized, i.e., cancerous (e.g., laryngeal papilloma, focal epithelial, cervical carcinoma).

In benign papillomavirus-induced diseases, the viral genome is maintained as a low-copy-number episome in the nucleus of basal cells of infected epithelium. It is believed that infection is maintained by the continued presence of low copy of viral DNA in basal cells, rather than by re-infection. Therefore, inhibition of viral DNA replication (through inhibition of the E1-E2 protein-protein binding interactions) can provide an opportunity for viral clearance from the epithelium.

The most common disease associated with HPV infection is the formation of warts, which are benign tumors generally caused by low-risk HPVs. They are usually self-limiting and naturally regress due to the influence of host immunological defenses. Host cellular mediated immune (CMI) responses are very important in modulating the course of HPV infections. When CMI responses are depressed due to pregnancy, HIV infection, or immunosuppressive therapy during organ transplant, the occurrence of HPV lesions increases.

Common warts (verrucae vulgaris) are almost universal in the population. They are most often found in children and young adults. Later in life the incidence of common warts decreases presumably due to immunologic and physiologic changes. Common warts are sharply demarcated, rough-surfaced, round or irregular, firm, and light gray, yellow, brown, or gray-black nodules of 2 to 10 mm in diameter. They appear most often on sites subject to trauma (e.g., fingers, elbows, knees, face) but may spread elsewhere. Periungual warts (around the nail plate) are common, as are plantar warts (verruca plantaris), which are flattened by pressure and surrounded by cornified epithelium. Mosaic warts are plaques formed by the coalescence of myriad of smaller, closely set plantar warts. Filiform warts are long, narrow growths usually found on the eyelids, face, neck, or lips. This morphologically distinctive variant of the common wart is benign and easy to treat. Flat warts (smooth, flat-topped, yellow-brown papules) are more common in children and young adults, found most often on the face, arms and legs and along scratch marks, and develop by autoinoculation. Variants of the common wart that are of unusual shape (e.g., pedunculated, or resembling a cauliflower) are most frequent on the head and neck, especially on the scalp and bearded region. Butcher's warts are mainly seen in people who frequently handle raw meat. An effective amount of an inhibitory agent of the invention can be administered to patients suffering from these benign forms of human papillomavirus disease to inhibit the growth of the wart(s) and/or to prevent its (their) recurrence. In certain embodiments, the inventive inhibitor is administered, in the appropriate formulation, directly to the area of the skin afflicted with the warty lesion(s).

The inventive methods and inhibitory agents can be applied to treat patients with epidermodysplasia verruciformis. Epidermodysplasia verruciformis (EV) is a rare, lifelong, autosomal recessive hereditary disorder affecting the skin. Widespread skin eruptions of flat-to-papillomatous, wart-like lesions and reddish-brown pigmented plaques on the trunk, hands, upper and lower extremities and the face are characteristic. This disease affects patients who are unable to resolve HPV-induced warts. The lesions may transform into malignant carcinomas, usually after the age of 30. Skin cancers appear initially on sun-exposed areas, such as the face and ear lobes. Patients with EV are usually infected with multiple types of HPV.

Ano-genital warts (also called venereal warts or condylomata acuminata) are flesh to gray in color, grow in mucous membranes, and vary in size from small, shiny papules, to large cauliflowerlike lesions. They can extend internally into the vagina and cervix, the rectal area, and inside the urethra. Most of these warts are benign and painless and many individuals completely clear HPV within weeks or months after infection. However, depending on the type of HPV and other ill defined co-factors, other outcomes are possible. These include: HPV persistence with no cellular abnormalities, transient cytological abnormalities that completely resolve, persistent cytological abnormalities, and cytological abnormalities that may progress to invasive cancer. The seriousness of ano-genital warts is underlined by the fact that HPV DNA is found in all grades of cervical intraepithelial neoplasia and that a subset of HPV types (the high-risk HPVs) is found in almost all cervical carcinomas.

According to the present invention, inhibitory agents that inhibit HPV DNA replication by precluding, reversing or disrupting the formation of the E1-E2 protein-protein complex, can be administered to a patient having one or more genital warts to reduce or inhibit the growth of the wart(s) and/or prevent its (their) recurrence. This method can be used to treat condyloma acuminata and/or flat cervical warts. In certain embodiments, the HPV infecting the patient is a high-risk HPV, including HPV-16, HPV-18, HPV-31, and HPV-33. Preferably, the high-risk HPV is HPV-16. In other embodiments, the HPV infecting the patient is a low-risk HPV.

In addition to being implicated in cervical cancer, human papillomaviruses are also believed to cause other ano-genital (vulvar and penile cancers) and epithelial malignancies such as laryngeal papillomas and focal epithelial hyperplasia.

Laryngeal papillomas occur on the vocal cords and laryngeal mucosa and, on rare occasions, may extend downwards into the trachea and bronchi. Two forms of the disease have been described. An adult form, usually non-aggressive and with solitary lesions and a male predilection, is often cured following a single operative procedure. However, some laryngeal papillomas in adults undergo malignant transformation; and heavy smoking has been determined to be a factor contributing to the transformation. Juvenile laryngeal papillomatosis, also called recurrent respiratory papillomatosis, is extremely aggressive and resistant to treatment. It typically involves the trachea, but may spread to the esophagus and bronchi, and rarely, to the lung where it actually destroys tissue, dramatically worsening the prognosis. It can also undergo malignant transformation. Infants with laryngeal papillomas are often born to mothers with infected genital condylomas of the vagina. Current strategies to prevent laryngeal papillomas in babies born to these women include eradication of the maternal lesions prior to delivery and delivery by Cesarian section.

According to the methods of treatment of the present invention, a patient suffering from laryngeal papillomas can be administered an inventive inhibitory agent, or a pharmaceutical composition thereof, so as to inhibit growth of the papillomas.

Focal epithelial hyperplasia (Heck's disease) is a highly contagious disease characterized by oral papillary lesions. Children are predominantly affected, but lesions may occur in young and middle-aged adults. Focal epithelial hyperplasia is somewhat different from other HPV infections in that it is able to produce extreme hyperplasia of the prickle cell layer of the epithelium with minimal production of surface projections or induction of connective tissue proliferation. The mucosa may be 8-10 times thicker than normal. Individual lesions are always broad based and multiple masses are scattered over a localized area. Lesions are frequently papillary in nature but relatively smooth-surfaces, flat-topped lesions are more commonly seen.

In other preferred embodiments, the inventive methods can also be used serially or in combination with chemotherapy, radiation, surgery, or other therapies with the goal of eliminating residual infected or pre-cancerous cells.

VI. Formulation, Dosage and Administration

The present invention also provides pharmaceutical compositions, which comprise, as active ingredient, an effective amount of at least one inhibitory agent, or a physiologically tolerable salt thereof. The pharmaceutical compositions of the invention may be formulated using conventional methods well known in the art. Such compositions include, in addition to the active ingredient(s), at least one pharmaceutically acceptable liquid, semiliquid or solid diluent acting as pharmaceutical vehicle, excipient or medium, and termed here “pharmaceutically acceptable carrier”.

According to the present invention, pharmaceutical compositions may include one or more inhibitory agents of the invention as active ingredients. Alternatively, a pharmaceutical composition containing an effective amount of one inventive inhibitor may be administered to a patient in combination with or sequentially with a pharmaceutical composition containing a different inventive inhibitory agent.

In another embodiment of this invention, an inhibitory agent, or a pharmaceutical composition thereof, may be administered serially or in combination with conventional therapeutics used in the treatment of HPV infections or diseases caused by them. Such therapeutics include interferons (IFN), such as IFN-γ, IFN-α and IFN-β derived from natural sources or produced by recombinant techniques; other cell mediators formed by leukocytes or produced by recombinant techniques such as, for example, interleukin-1, interleukin-2, tumor necrosis factor, macrophage colony stimulating factor, macrophage migration inhibitory factor, macrophage activation factor, lymphotoxin and fibroblast growth factor. Inhibitory agents of the invention may also be co-administrated with other anti-viral agents such as acyclovir, gancyclovir, vidarabidine, foscarnet, cidofovir, amantidine, ribavirin, zidovudine, didanosine, trifluorothymidine, or zalcitabine.

Alternatively or additionally, an inventive inhibitory agent, or a pharmaceutical composition thereof, may be administered serially or in combination with conventional therapeutic regimens for HPV infection such as, for example, surgery and necrotization by cryo-, electro- or laser cauterization. Such combination therapies may present the advantage of avoiding the potential toxicity or risks associated with those therapies.

The treatment may consist of a single dose or a plurality of doses over a period of time. An inhibitory agent or pharmaceutical composition of the invention may also be released from a depot form per treatment. The administration may be carried out in any convenient manner such as by injection (subcutaneous, intravenous, intramuscular, intraperitoneal, or the like), oral administration, sublingual administration, or topical application to exert local therapeutic effects. In a preferred embodiment, the inventive inhibitory agent, or a pharmaceutical composition thereof, is topically applied on the area of the skin afflicted with wart(s)

Effective dosages and administration regimens can be readily determined by good medical practice and the clinical condition of the individual patient. The frequency of administration will depend on the pharmacokinetic parameters of the agents and the route of administration. The optimal pharmaceutical formulation can be determined depending upon the route of administration and desired dosage. Such formulations may influence the physical state, stability, rate of in vivo release, and rate of in vivo clearance of the administered agents. Depending on the route of administration, a suitable dose may be calculated according to body weight, body surface area, or organ size. Optimization of the appropriate dosage can readily be made by those skilled in the art in light of pharmacokinetic data observed in human clinical trials. The final dosage regimen will be determined by the attending physician, considering various factors which modify the action of drugs, e.g., the drug's specific activity, the severity of the damage and the responsiveness of the patient, the age, condition, body weight, sex and diet of the patient, the severity of any infection, time of administration and other clinical factors. As studies are conducted, further information will emerge regarding the appropriate dosage levels and duration of treatment for various diseases and conditions.

The dose and administration regime will also be a function of whether the inhibitory agents are being administered therapeutically or prophylactically. Typically, the amount of peptide administered per dose will be in the range of about 0.1 to 25 mg/kg of body weight, with the preferred dose being about 0.1 to 10 mg/kg of patient body weight.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Example 1 relates to a binding assay used to identify small molecules capable of preventing or disrupting the formation of the HPV-16 E1-E2 complex. The assay, which was carried out to screen a small molecule library of 1,3-dioxanes, led to the identification of compound 1, along with nine other “leads”. Example 2 describes the synthesis of compounds 2 and 3, which are two enantiomers of a derivative of compound 1. Example 3 illustrates the determination by surface plasmon resonance of the equilibrium dissociation constants for the binding of compound 2 and compound 3 to the HPV-16 E2 protein. Example 4 describes biological assays that can be used to demonstrate the disruption of the E1-E2 protein-protein binding induced by compounds 2 and 3 in vitro.

### Example 1

### E2 Binding Screening Assay

An assay for the identification of E2 ligands was used to screen a 1,3-Dioxane small molecule library which was developed in the Applicants' laboratory and whose synthesis has previously been described in great detail (S. M. Sternson et al., J. Am. Chem. Soc. 2001, 123: 1740-1747, which is incorporated herein by reference in its entirety). The assay was performed according to a method developed in the Applicants' laboratory (G. MacBeath et al., J. Am. Chem. Soc. 1999, 121: 7967-7968, which is incorporated herein by reference in its entirety).

Two 23-mer HPV-16 E2 peptides (corresponding to amino acids 28-50 of the full-length native HPV-16 E2 protein) were used in the screen. The amino acid sequence of the interacting peptide is: A-H-I-D-Y-W-K-H-M-R-L-E-C-A-I-Y-Y-K-A-R-E-M-G, and the amino acid sequence of the specificity peptide, which contains an alanine substitution for glutamic acid at position 39 (E39A), is: A-H-I-D-Y-W-K-H-M-R-L-A-C-A-I-Y-Y-K-A-R-E-M-G. The two polypeptides were labeled with Cy5 fluorescent dye (Amersham Biosciences Inc., Piscataway, N.J.), by attaching the carbonyl group of Cy5 to the amino-terminus of the peptide (Charles Dahl, Biopolymers Laboratory, Harvard Medical School). The peptides were dissolved in DMSO at a concentration of 10 mM.

The 1,3-dioxane small molecule library was printed onto microscope slides and stored frozen at −70° C. Before use, the library was thawed to room temperature and blocked with PBST (i.e., phosphate buffer saline containing 0.1% Tween-20, and 3% BSA) for 30 minutes. The slide was then washed in PBST 3 times and incubated with the interacting or specificity polypeptide at a concentration of 200 μM in PBST for 30 minutes. After incubation, the slide was washed in PBST twice for 3 minutes to remove any unbound polypeptide and then allowed to dry.

The detection of the fluorescence emitted by the bound polypeptide was performed by scanning the slide using an ArrayWoRx Biochip Reader (Applied Precision, Issaquah, Wash.) at a resolution of 5 μm per pixel. Double filters for both incident and emitted lights (Cy5/Cy5 excitation/emission) were used. Small molecules that were found to bind to the interacting peptide and not to the specificity peptide were identified and used in subsequent screens. The screening of the 1,3-dioxane library, for example, led to the identification to compound 1 (the chemical structure of which is presented in FIG. 1) along with nine other leads.

### Example 2

### Synthesis of Compounds 2 and 3

Compounds 2 and 3 were synthesized with the goal of developing molecules with improved solubility properties compared with compound 1.

Resin Starting Materials. The syntheses of compounds 2 and 3 were carried out by solid phase organic chemistry using two different resins (resins A1 and B1) as starting materials. The chemical structures of resins A1 and B1 are presented below. Detailed synthetic procedures for the preparation of these resins have been described (S. M. Sternson et al., Org. Let., 2001, 3: 4239-4242, which is incorporated herein by reference in its entirety).

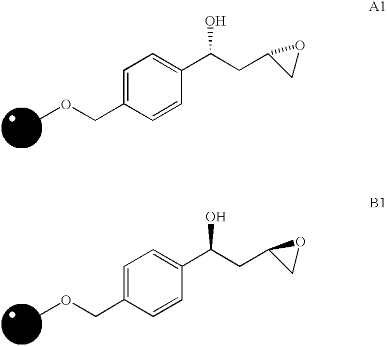

Procedure. The synthesis of compound 2 is illustrated by the scheme presented below.

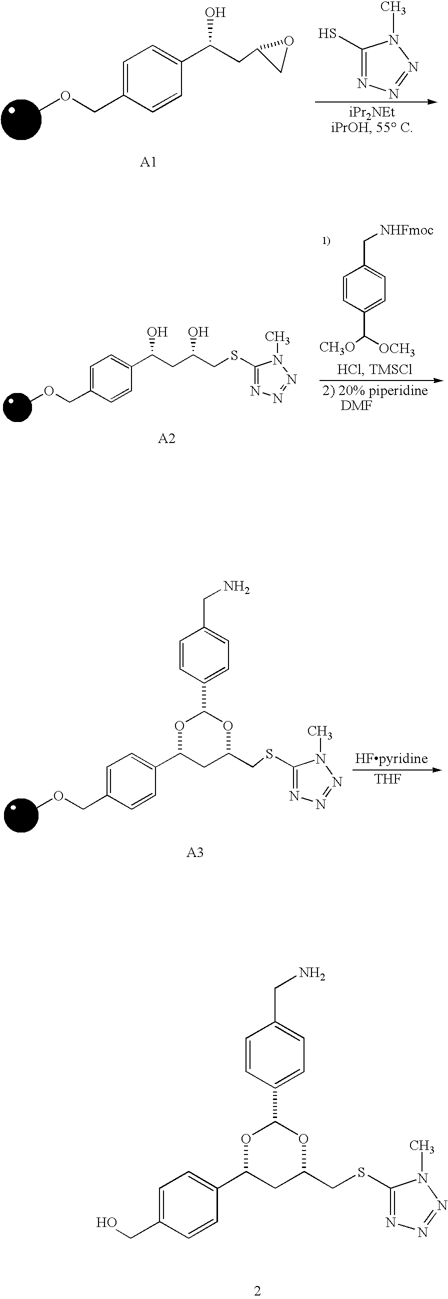

To resin A1 (20 mg) in a 4 mL-Wheaton vial was added 5-mercapto-1-methyltetrazole (35 mg, 0.30 mmol) followed by isopropanol (0.3 mL) and diisopropylethylamine (0.051 mL, 0.30 mmol). The vial was flushed with argon, capped, and allowed to stand in an oven at 55° C. for 24 h. The reaction mixture was filtered and washed with dimethylformamide (DMF) (3×10min), tetrahydrofuran (THF) (3×10min), and dichloromethane (CH₂Cl₂) (3×10 min) to give the 1,3-diol resin A2. A2 was then treated with Fmoc-amino dimethylacetal (the synthesis of which has previously been described by S. M. Sternson et al. Org. Let., 2001, 3: 4239-4242, which is incorporated herein by reference in its entirety) in a solution of 0.05 M HCl in anhydrous 1,4-dioxane (0.6 mL) and chlorotrimethylsilane (TMSCl) (0.03 mL, 0.24 mmol).

After 4 hours, the reaction was quenched with anhydrous pyridine (0.1 mL), and the reaction mixture was filtered and washed with DMF (3×10 min) and THF (3×10 min). The resin was then immediately subjected to 20% piperidine/DMF (1×10 min.; 1×20 min.; 1×10 min.) with THF washes (3 min.) in between treatments. The resin was then washed with DMF (3×10 min), THF (3×10 min), and CH₂Cl₂ (3×10 min) to give the 1,3-dioxane resin A3. Crude compound 2 was obtained after resin cleavage with 18:1:1 THF:pyridine:HF.pyridine. This material was purified by flash column chromatography using a 3:30:70 triethylamine:methanol:ethyl acetate mixture as mobile phase.

The overall yield of the preparation of compound 2 was 58%. ¹H NMR of compound 2 (500 MHz, CD₃OD) gave: δ 7.40-7.31 (m, 8H), 5.73 (s, 1H, J=7.3), 4.96 (dd, 1H, J=11.2 Hz, 2.0 Hz), 4.57 (s, 2H), 4.37 (m, 1H), 3.90 (s, 3H), 3.84 (s, 2H), 3.62 (dd, 1H, J=13.9 Hz, 3.9 Hz), 3.48 (dd, 1H, J=13.9 Hz, 7.8 Hz), 2.04 (dt, 1H, J=13.2 Hz, 2.0 Hz), 1.76 (dt, 1H, J=13.2 Hz, 11.2 Hz). APCI/MS analysis led to (M+H⁺)=428 as expected.

Compound 3 was synthesized in a similar way using resin B1 as starting material. The overall yield of the preparation of compound 3 was 62%. ¹H NMR of compound 3 (500 MHz, CD₃OD) gave: δ 7.40-7.31 (m, 8H), 5.73 (s, 1H, J=7.3), 4.96 (dd, 1H, J=11.2 Hz, 2.0 Hz), 4.57 (s, 2H), 4.37 (m, 1H), 3.90 (s, 3H), 3.84 (s, 2H), 3.62 (dd, 1H, J=13.9 Hz, 3.9 Hz), 3.48 (dd, 1H, J=13.9 Hz, 7.8 Hz), 2.04 (dt, 1H, J=13.2 Hz, 2.0 Hz), 1.76 (dt, 1H, J=13.2 Hz, 11.2 Hz). APCI/MS analysis led to (M+H⁺)=428 as expected.

### Example 3

### Determination of Dissociation Constants by Surface Plasmon Resonance

Surface Plasmon Resonance Instrument. Surface plasmon resonance experiments were performed with a Biacore® 3000 Biosensor (Biacore AB, Uppsala, Sweden). To keep the instrument in good working condition, a series of washing steps were performed on a weekly basis. In addition to the prescribed maintenance washes, the fluidic system was primed three times in a row with the following reagents: 0.5% (w/v) SDS, 6 M urea, 1% (v/v) acetic acid, and 0.2 M NaHCO₃. When not in use, a standby program injects water at a flow rate of 5 μL/minute to prevent salt build-up in the lines or integrated fluidic cartridge. Whenever running buffer was changed, the system was primed three times in order to equilibrate the sensor chip and the instrument.

Immobilization of anti-GST Antibody. A CM5 sensor chip was docked and the system was primed three times with filtered and degassed PBST containing 5% (v/v) DMF. Affinity purified and sterile filtered goat-anti-GST IgG (0.8 mg/mL in 150 mM NaCl) from the Biacore GST kit for fusion capture was diluted to 30 μg/mL in 10 mM sodium acetate, pH 5.0 (100 μL per immobilization). Two flow cells were activated with 1:1 NHS:EDC (N-hydroxysuccinimide:ethyl-2-(dimethylaminopropyl)carbodiimide) for 8 minutes at a flow rate of 5 μL/min using the “surface preparation: immobilization: amine coupling” wizard application. The antibody solution was applied to each surface for 7 minutes at the same flow rate followed by deactivation with ethanolamine for 7 minutes. A typical immobilization level using these conditions at 25° C. is 10,000 RU. Depending on the frequency of use, sensor chips with flow cells containing coupled anti-GST were stored at 4° C. under humid conditions for up to 1 month.

Capture of GST-E2 Fusion Protein in Anti-GST Flow Cells. Recombinant GST (Schistosoma japonicum) was immobilized as a control, typically in flow cell 1 or 3, for antibody immobilization and small molecule binding. Sterile filtered GST (0.2 mg/mL in HBS buffer-10 mM Hepes pH 7.4, 150 mM NaCl, 3 mM EDTA, and 0.005% (v/v) P20 surfactant) was diluted to 5 μg/mL in HBS buffer (50 μL per capture). The protein solution was injected, using the “quickinject” option, at 5 μL/min for 7 minutes. Typical capture responses ranged between 1,000 and 1,500 RU with dissociation rates less than 3 RU/min. The E2-GST fusion proteins were diluted to 5-10 μg/mL in HBS buffer (60 μL per capture). The solution was injected at 5 μL/min for 10 minutes onto the active sample flow cell, typically flow cell 2 or 4. Captured responses ranged between 900 and 2,000 RU with dissociation rates less than 3 RU/min.

Small Molecule Sample Preparation and Injection. Surface plasmon resonance experiments were performed with a Biacore® 3000 essentially as recommended by Biocore AB. For each compound, serial dilutions were prepared over a wide concentration range: 1 μM, and 500, 250, 125, 62.5, 31.3, 15.6, 7.8, 3.9, 2, 1 and 0 nM. All compounds were warmed up to room temperature prior to dilution. Dilutions were prepared in DMF such that 4 μL of compound solution were added to 196 μL warm PBST buffer to give a final DMF concentration of 2% (v/v) in each sample. The running buffer for all experiments was PBST containing 2% (v/v) DMF. Samples were centrifuged to eliminate any precipitate. Six PBS solutions were prepared with the following concentrations of DMF (% v/v): 4.0, 3.0, 2.5, 2.0, 1.5 and 1.0. Blank injections, containing 2% (v/v) DMF, were also performed between samples to ensure that the needle, tubing, and flow-path were not contaminated with compound(s) from the previous injection(s).

Samples were setup for automated injection using the “binding analysis” application wizard. The flow rate was set at 50 μL/min and each sample was injected for 3 minutes. Each injection was followed by a dissociation period, of usually 1 minute, in which buffer was passed through the cells at 50 μL/min. Different concentrations were injected in a random order. For each experiment, a % cosolvent calibration was performed. The change in response units was recorded for both the control and active flow cells during any given injection (including blanks). These values were saved as “report point tables” and analyzed in Microsoft Excel. To construct the % cosolvent calibration curve, [RU_{active}−RU_{control}] (y-axis) was plotted against RU_{control} (x-axis) and a linear fit was performed. The equation was used to correct sample values, corresponding to small molecule injections, for bulk effects by entering RU_{control} values for x. The equation was solved for y (correction factor). The correction factor was then subtracted from [RU_{active}−RU_{control}] for each sample to give the corrected RU value. For each corrected data set, RU (y-axis) was plotted against concentration (x-axis). Curves were imported into the BioEvaluation software and a steady state fit was performed.

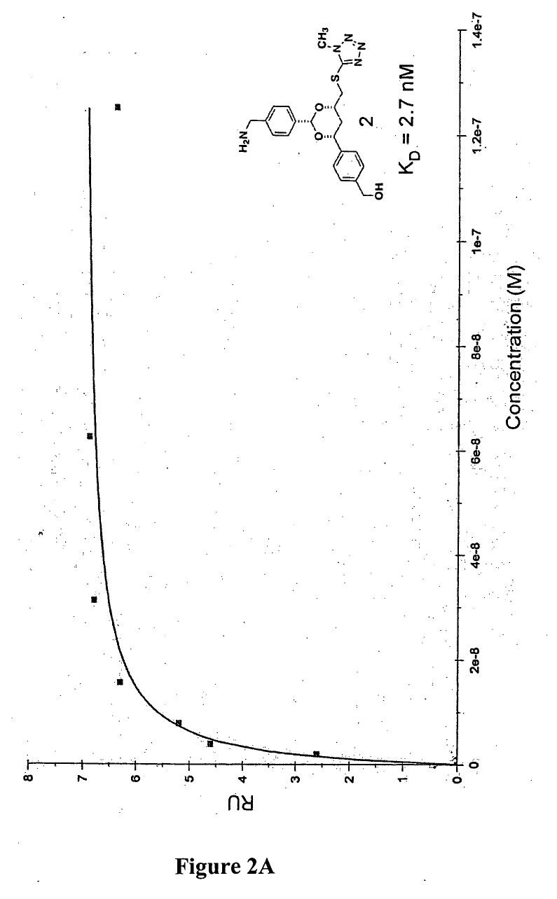

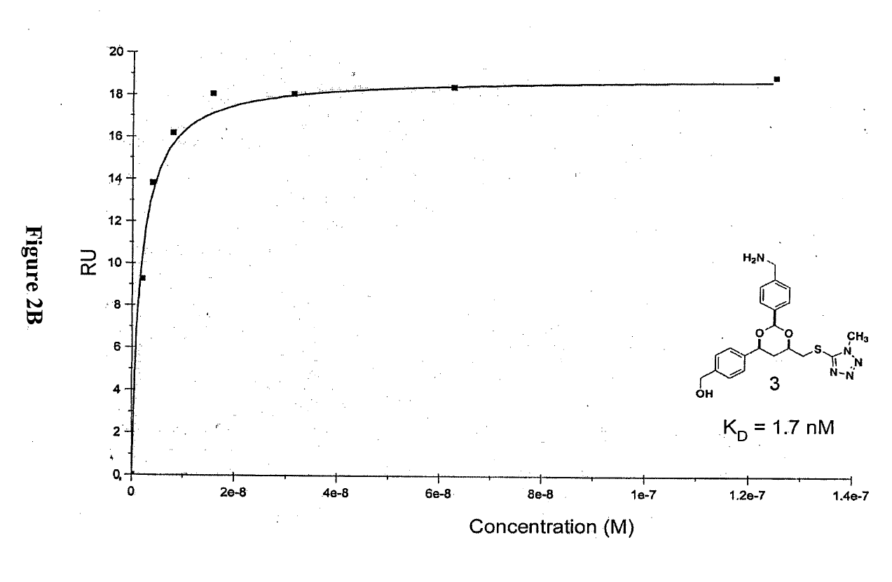

FIG. 2 shows the plots obtained by surface plasmon resonance for the binding of compounds 2 and 3 to HPV-16 E2. Using these plots and a steady state model, the dissociation constant was determined to be 2.7 nM for compound 2 and 1.7 nM for compound 3.

### Example 4

### Disruption of E1-E2 Protein-Protein Binding in vitro

Different methods may be used to determine whether anti-viral candidates are able to disrupt the E1-E2 protein-protein interaction in vitro.

The surface plasmon resonance approach involves capturing either partner on a CM5 chip (Biacore) that has been covalently modified with an antibody for affinity capture (anti-GST, anti-FLAG, anti-EE) as described in Example 3. The partner protein is then injected in solution, which causes the formation of the complex at the surface of the CM5 chip, as indicated by an increase in response units. The small molecule candidate is then injected at a concentration of 1 μM. A disruption of the protein-protein complex due to the presence of the small molecule is indicated by a decrease in response units.

Another method is fluorescence anisotropy, which monitors the rate of tumbling of the components that make up the complex in solution. This approach requires that a fluorescently labeled E2 protein be prepared. The formation of the E1-E2 protein-protein complex as well as its disruption by addition of small molecule candidates may then be monitored. Alternatively, E2 may be pre-treated with a small molecule candidate to see if it prevents formation of the E1-E2 complex.

Another in vitro assay that tests the ability of small molecules to prevent the formation of the E1-E2 complex involves immunoprecipitation of E1 and western blotting for E2. Cellular extracts from insect cells infected with baculoviruses that contain the cDNA of E1 and E2 are isolated. The E1 protein is tagged with EE, and the E2 protein is untagged. 300-500 ng of EE-E1 and 80-120 ng of E2 extract are incubated overnight at 4° C. with anti-EE monoclonal antibody and protein G in the presence or absence of (5 nM to 50 μM of) a small molecule in NET buffer (50 mM Tris-HCl, pH 7.5, 150 mM NaCl, 0.1% NP-40, 1 mM EDTA pH 8.0, 0.25% gelatin). The protein G beads are then spun down and washed 3 times in NET, and samples are analyzed by SDS-PAGE and western blots are stained with anti-E2 antibody. The western blot for E2 indicates if the small molecule tested is able to prevent E1 from binding and immunoprecipitating E2. In this assay, non-binding compounds are used as controls.

1. A screening system including an interacting peptide and a specificity peptide, wherein the interacting peptide comprises a portion of a viral interacting protein that is sufficient to allow the interacting peptide to bind to the interacting protein's partner, and wherein the specificity peptide comprises an identical portion of the viral interacting protein except that the specificity peptide includes a mutation or alteration that reduces or destroys its ability to bind to the interacting protein's partner.

2. The screening system of claim 1, wherein binding interactions between the viral interacting protein and the interacting protein's partner are important in the life cycle of a papillomavirus.

3. The screening system of claim 2, wherein the papillomavirus is a human papillomavirus (HPV).

4. The screening system of claim 3, wherein the human papillomavirus is a low-risk HPV.

5. The screening system of claim 3, wherein the human papillomavirus is a high-risk HPV.

6. The screening system of claim 5, wherein the high-risk HPV is selected from the group consisting of HPV-16, HPV-18, HPV-31 and HPV-33.

7. The screening system of claim 5, wherein the high-risk HPV is HPV-16.

8. The screening system of claim 1, wherein the viral interacting protein is a HPV E1 or HPV E2 protein.

9. The screening system of claim 1, wherein the viral interacting protein is HPV-16 E2 protein and the interacting protein's partner is HPV-16 E1 protein.

10. The screening system of claim 1, wherein the interacting peptide and specificity peptide are fluorescently labeled.

11. A screening system including an interacting peptide and a specificity peptide, wherein the interacting peptide comprises a portion of HPV-16 E2 protein that is sufficient to allow the interacting peptide to bind to HPV-16 E1 protein, and wherein the specificity peptide comprises an identical portion of HPV-16 E2 protein except that the specificity peptide includes a mutation or alteration that reduces or destroys its ability to bind to HPV-16 E1 protein.

12. The screening system of claim 11, wherein the interacting peptide comprises a portion of HPV-16 E2 protein flanking the E39 residue, and wherein the mutation or alteration that reduces or destroys the ability of the specificity peptide to bind to HPV-16 E1 protein is a mutation or alteration of the E39 residue.

13. The screening system of claim 11, wherein the interacting peptide and specificity peptide are fluorescently labeled.

14. The screening system of claim 12, wherein the interacting peptide is a 23-mer with the following amino acid sequence:
A-H-I-D-Y-W-K-H-M-R-L-E-C-A-I-Y-Y-K-A-R-E-M-G,
and the specificity peptide is a 23-mer with the following amino acid sequence:
A-H-I-D-Y-W-K-H-M-R-L-A-C-A-I-Y-Y-K-A-R-E-M-G.

15. The screening system of claim 14, wherein the interacting peptide and specificity peptide are fluorescently labeled with Cy5.

16. A method for identifying an anti-viral agent comprising steps of:
providing a collection of candidate agents;
contacting a candidate agent with an interacting peptide comprising a portion of a viral interacting protein that is sufficient to allow the interacting peptide to bind to the interacting protein's partner;
contacting the candidate agent with a specificity peptide comprising an identical portion of the viral interacting protein except that the specificity peptide includes a mutation or alteration that reduces or destroys its ability to bind to the viral interacting protein's partner;
determining the relative binding; and
identifying the candidate agent as an inhibitory agent based upon its ability to preferably bind to the interacting peptide as compared to the specificity peptide.

17. The method of claim 16, wherein binding interactions between the viral interacting protein and the interacting protein's partner are important in the life cycle of a papillomavirus.

18. The method of claim 17, wherein the papillomavirus is a human papillomavirus (HPV).

19. The method of claim 18, wherein the human papillomavirus is a low-risk HPV.

20. The method of claim 18, wherein the human papillomavirus is a high-risk HPV.

21. The method of claim 20, wherein the high-risk HPV is selected from the group consisting of HPV-16, HPV-18, HPV-31 and HPV-33.

22. The method of claim 20, wherein the high-risk HPV is HPV-16.

23. The method of claim 16, wherein the viral interacting protein is a HPV E1 or HPV E2 protein.

24. The method of claim 16, wherein the viral interacting protein is HPV-16 E2 protein and the interacting protein's partner is HPV-16 E1 protein.

25. The method of claim 16, wherein the interacting peptide and specificity peptide are fluorescently labeled and the relative binding is determined by fluorescence.

26. The method of claim 16, wherein the collection of candidate agents is a library of small molecules.

27. A method for identifying an anti-viral agent comprising steps of:
providing a collection of candidate agents;
contacting a candidate agent with an interacting peptide comprising a portion of HPV-16 E2 protein that is sufficient to allow the interacting peptide to bind to HPV-16 E1 protein;
contacting the candidate agent with a specificity peptide comprising an identical portion of HPV-16 E2 protein except that the specificity peptide includes a mutation or alteration that reduces or destroys its ability to bind to HPV-16 E1 protein;
determining the relative binding; and
identifying the candidate agent as an inhibitory agent based upon its ability to preferably bind to the interacting peptide as compared to the specificity peptide.

28. The method of claim 27, wherein the interacting peptide comprises a portion of HPV-16 E2 protein flanking the E39 residue, and wherein the mutation or alteration that reduces or destroys the ability of the specificity peptide to bind HPV-16 E1 protein is a mutation or alteration of the E39 residue.

29. The method of claim 27, wherein the interacting peptide is a 23-mer with the following amino acid sequence:
A-H-I-D-Y-W-K-H-M-R-L-E-C-A-I-Y-Y-K-A-R-E-M-G,
and the specificity peptide is a 23-mer with the following amino acid sequence:
A-H-I-D-Y-W-K-H-M-R-L-A-C-A-I-Y-Y-K-A-R-E-M-G.

30. The method of claim 27, 28 or 29, wherein the interacting peptide and specificity peptide are fluorescently labeled, and the relative binding is determined by fluorescence.

31. The method of claim 27, 28 or 29, wherein the collection of candidate agents is a library of small molecules.

32. An inhibitory agent identified by the method of claim 16 or 17, wherein said inhibitory agent is a small molecule.

33. An inhibitory agent identified by the method of claim 27, 28 or 29, wherein said inhibitory agent is a small molecule.

34. A pharmaceutical composition comprising an effective amount of at least one inhibitory agent of claim 32, or a physiologically tolerable salt thereof, and at least one pharmaceutically acceptable carrier.

35. A pharmaceutical composition comprising an effective amount of at least one inhibitory agent of claim 33, or a physiologically tolerable salt thereof, and at least one pharmaceutically acceptable carrier.

36. A method for reducing or inhibiting viral replication in a system, the method comprising contacting the system with an effective amount of an inhibitory agent identified by the method of claim 16.

37. The method of claim 36, wherein the inhibitory agent is a small molecule that reduces or inhibits viral replication by inhibiting protein-protein interactions that are important for the life cycle of a papillomavirus.

38. The method of claim 37, wherein the papillomavirus is a human papillomavirus (HPV).

39. The method of claim 38, wherein the human papillomavirus is a low-risk HPV.

40. The method of claim 38, wherein the human papillomavirus is a high-risk HPV.

41. The method of claim 40, wherein the high-risk HPV is selected from the group consisting of HPV-16, HPV-18, HPV-31 and HPV-33.

42. The method of claim 40, wherein the high-risk HPV is HPV-16.

43. The method of claim 37, wherein the protein-protein interactions that are important for the life cycle of a papillomavirus involve a HPV E1 and HPV E2 proteins.

44. The method of claim 37, wherein the protein-protein interactions that are important for the life cycle of a papillomavirus involve HPV-16 E1 protein and HPV-16 E2 protein.

45. A method for reducing or inhibiting the viral replication of HPV-16 in a system, the method comprising contacting the system with an effective amount of an inhibitory agent identified by the method-of claim 27, 28 or 29.

46. The method of claim 45, wherein the inhibitory agent is a small molecule that reduces or inhibits viral replication by inhibiting binding interactions between HPV-16 E2 protein and HPV-16 E1 protein.

47. A method for reducing or inhibiting viral replication of a papillomavirus in a system, the method comprising contacting the system with an effective amount of an inhibitory agent with the following chemical structure:

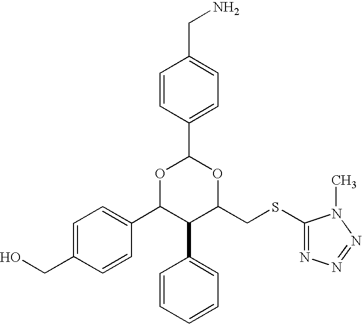

48. A method for reducing or inhibiting viral replication of a papillomavirus in a system, the method comprising contacting the system with an effective amount of an inhibitory agent with the following chemical structure:

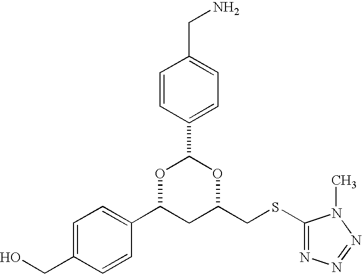

49. A method for reducing or inhibiting viral replication of a papillomavirus in a system, the method comprising contacting the system with an effective amount of an inhibitory agent with the following chemical structure:

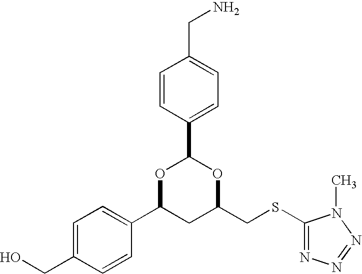

50. A method for treating a disease or medical condition associated with a virus, the method comprising administering to an individual in need thereof an effective amount of an inhibitory agent identified by the method of claim 16.

51. The method of claim 50, wherein the inhibitory agent is a small molecule that reduces or inhibits viral replication by inhibiting protein-protein interactions that are important for the life cycle of a papillomavirus.

52. The method of claim 51, wherein the papillomavirus is a human papillomavirus (HPV).

53. The method of claim 52, wherein the human papillomavirus is a low-risk HPV.

54. The method of claim 52, wherein the human papillomavirus is a high-risk HPV.

55. The method of claim 54, wherein the high-risk HPV is selected from the group consisting of HPV-16, HPV-18, HPV-31 and HPV-33.

56. The method of claim 54, wherein the high-risk HPV is HPV-16.

57. The method of claim 54, wherein the high risk HPV is associated with cervical dysplasia or cervical cancer.

58. The method of claim 51, wherein the protein-protein interactions that are important for the life cycle of a papillomavirus involve HPV E1 and HPV E2 proteins.

59. The method of claim 51, wherein the protein-protein interactions that are important for the life cycle of a papillomavirus involve HPV-16 E1 protein and HPV-16 E2 protein.

60. A method for treating a disease or medical condition associated with HPV-16, the method comprising administering to an individual in need thereof an effective amount of an inhibitory agent identified by the method of claim 27, 28 or 29.

61. The method of claim 60, wherein the inhibitory agent is a small molecule that reduces or inhibits viral replication of HPV-16 by inhibiting binding interactions between HPV-16 E1 protein and HPV-16 E2 protein.

62. The method of claim 60, wherein HPV-16 is associated with cervical dysplasia or cervical cancer.

63. A method for treating a disease or medical condition associated with a papillomavirus, the method comprising administering to an individual in need thereof an effective amount of an inhibitory agent with the following chemical structure:

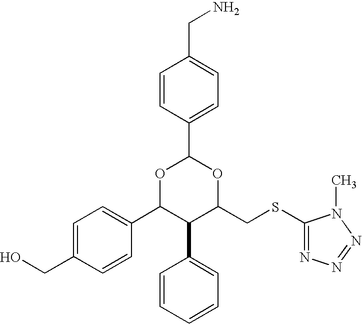

64. A method for treating a disease or medical condition associated with a papillomavirus, the method comprising administering to an individual in need thereof an effective amount of an inhibitory agent with the following chemical structure:

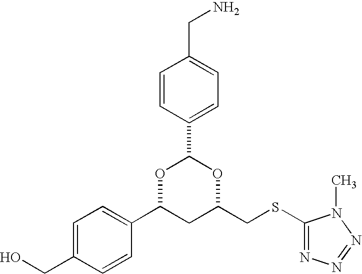

65. A method for treating a disease or medical condition associated with a papillomavirus, the method comprising administering to an individual in need thereof an effective amount of an inhibitory agent with the following chemical structure: [chemical structure]


SEQUENCE LISTING (2 sequences)

SEQ ID NO: 1 - protein, 23 aa, Artificial
  Human
AHIDYWKHMRLECAIYYKAREMG

SEQ ID NO: 2 - protein, 23 aa, Artificial
  Human
AHIDYWKHMRLACAIYYKAREMG